(19) United States
(12) Patent Application Publication
(10) Pub. No.: US 2026/0120855 A1
(43) Pub. Date: Apr. 30, 2026

(54) CLINICAL WORKFLOW OPTIMIZATION SYSTEM VIA GENERATIVE ARTIFICIAL INTELLIGENCE AND METHOD THEREOF

(71) Applicants: Chung Shan Medical University, Taichung City (TW); Chung Shan Medical University Hospital, Taichung City (TW)

(72) Inventors: Wei-Chih SHEN, Taichung City (TW); Da-Yi YAN, Chiayi County (TW)

(21) Appl. No.: 19/366,983

(22) Filed: Oct. 23, 2025

Related U.S. Application Data
(60) Provisional application No. 63/713,139, filed on Oct. 29, 2024.

(51) Int. Cl. G16H 40/20; G06Q 10/0633

(52) U.S. Cl. CPC G16H 40/20; G06Q 10/06334

(57) ABSTRACT

A clinical workflow optimization system includes a data processing and analysis platform electrically connected or communicatively coupled to a user device and an HIS server, and a generative AI model set communicatively coupled to the data processing and analysis platform. The data processing and analysis platform provides one or more assistant tools. When the user device selects an assistant tool to execute a task instruction, the data processing and analysis platform uses one model of the generative AI model set to interpret and execute the task instruction. The analysis platform presents the execution result of the task instruction to the user device, wherein data required to execute the task instructions is recorded in the content of the task instructions or provided by the HIS server, the user device, or an external database. On the platform, users can independently develop and build clinical data processing tools using natural language.

### BACKGROUND

### Field of the Disclosure

The present disclosure relates to the technical field of workflow optimization and, more particularly, to a clinical workflow optimization system via generative artificial intelligence and a method thereof.

### Description of Related Art

In the clinical workflow of medical institutions (for example, hospitals), the creation and maintenance of data and organizational documents are crucial to the operation of medical information systems. However, medical staff (including personnel from various roles in medical institutions) must utilize fragmented periods to search, transcribe, compile, and interpret data and files scattered across different information systems before drafting documents. These extensive, tedious, highly repetitive, and time-sensitive tasks severely reduce the time available for professional work, becoming heavy burdens that contribute to the deterioration of the working environment.

Medical information systems in medical institutions are often highly customized, resulting in significant differences between hospital systems. Consequently, data interoperability between different hospitals is challenging. Additionally, the lack of standardized and unified interfaces hinders AI integration and limits the potential of AI to improve the medical environment.

In addition, even within the same medical institution, each department has specific requirements for processing and analyzing clinical data and organizational documents. Existing solutions typically only address a single clinical need, making it difficult to meet these diverse needs comprehensively.

Improving and optimizing clinical workflows requires collaboration among medical personnel, information technology personnel, and artificial intelligence engineers. However, communication and coordination between personnel of different job categories often experience gaps and difficulties. Due to considerations of development scale, cost, and benefits, it is challenging to take into account the niche professional needs of specific job categories.

Therefore, a clinical workflow optimization system and method via generative artificial intelligence is needed to alleviate and/or obviate the above problems.

### SUMMARY

The present disclosure provides a clinical workflow optimization system, which includes: a data processing and analysis platform, and a generative artificial intelligence model set. The data processing and analysis platform is communicatively coupled to a user device and a hospital information system server for providing one or more configured assistant tools. The generative artificial intelligence model set includes at least one generative artificial intelligence model electrically connected or communicatively coupled to the data processing and analysis platform. When the user device selects one of the assistant tools to execute a task instruction, the data processing and analysis platform interprets the content of the task instruction through at least one generative artificial intelligence model. It executes the task instruction, and the data processing and analysis platform presents an execution result of the task instruction to the user device, wherein data required to execute the task instruction is recorded in the content of the task instruction, or comes from the hospital information system server, the user device, or an external database.

In some embodiments, the present disclosure provides a clinical workflow optimization system having the following features: (1) a data processing and analysis platform may be communicatively coupled to a user device and a hospital information system (HIS) server to receive and integrate a variety of clinical data, including structured data (such as medical record fields) and unstructured data (such as text records, voice, images, audio and video, etc.), and support synchronous or asynchronous acquisition; (2) a generative artificial intelligence model set may include at least one generative AI model with specific functions, such as summary generation, data structuring, semantic classification, translation conversion, or speech recognition; the model set is electrically connected or communicatively coupled to the data processing and analysis platform; the system receives data processing instructions input by the user in natural language, automatically identifies the data and models required for the processing task, and performs data analysis and outputs results.

In some embodiments, the data processing and analysis platform may further provide a standardized and modular “assistant” creation tool. The assistant is a data processing task module that allows users to customize the following elements through a graphical or natural language interface: (1) data source and type (for example, HIS fields, user photos, voice input, etc.); (2) data screening conditions and pre-processing logic (for example, field filtering, data time interval, etc.); (3) generative artificial intelligence model and execution parameters to be used; (4) processing result storage method (for example, format, fields, storage location) and subsequent processing flow.

In some embodiments, the assistant module may be stored in a platform database and support version control and cross-system migration, making it reusable and portable. Furthermore, the assistant module may be packaged as a mobile application (App), such as a SMART on FHIR-compliant application (SMART on FHIR App), which supports clinical information systems embedded in FHIR architecture, but not limited to FHIR databases, thereby enhancing its portability, interoperability, and deployment flexibility.

In some embodiments, when a user selects a configured assistant and issues a task instruction through a user device, the data processing and analysis platform automatically invokes the corresponding AI model based on the assistant module settings to perform the task. This includes interpreting the processing instruction, obtaining data sources (such as HIS, device, or external database), data pre-processing, model inference, and result formatting. After processing is completed, the system instantly displays the results on the user's device. It may be stored in a designated location or trigger subsequent processing steps based on the settings.

The present disclosure also provides a clinical workflow optimization method, which is executed by a clinical workflow optimization system. The system comprises a data processing and analysis platform, as well as a generative artificial intelligence model set. The generative artificial intelligence model set includes at least one generative artificial intelligence model. The method includes the steps of: communicatively coupling the data processing and analysis platform to a user device and a hospital information system server; electrically connecting or communicatively coupling the data processing and analysis platform to the generative artificial intelligence model set; enabling the data processing and analysis platform to provide one or more configured assistant tools; when the user device selects one of the assistant tools to execute a task instruction, the data processing and analysis platform interpreting content of the task instruction through the at least one generative artificial intelligence model and executing the task instruction, wherein data required to execute the task instruction is recorded in the content of the task instruction, or comes from the hospital information system server, the user device or an external database; and enabling the data processing and analysis platform to present an execution result of the task instruction to the user device.

In some embodiments, the clinical workflow optimization method of the present disclosure has the following steps: (1) establishing and running a data processing and analysis platform, which is communicatively coupled to the user device and the HIS server; (2) integrating a set of generative artificial intelligence models in the platform, wherein the model has various processing functions, and is communicatively coupled to the platform; (3) building an assistant module by the user through graphical or natural language, and settings the processing logic and process conditions; (4) when the user selects the assistant and issues a task instruction, using the platform to set the invoked model according to the assistant setting to execute the processing task; (5) during the execution process, using the system to dynamically invoke the data source, including HIS data, user-uploaded content, or external medical databases; (6) transmitting the analysis and processing results back to the user device for being stored, subsequently triggered, or interactively verified according to user needs.

Other novel features of the disclosure will become more apparent from the following detailed description when taken in conjunction with the accompanying drawings.

### BRIEF DESCRIPTION OF DRAWINGS

FIG. 1 is a system architecture diagram of a clinical workflow optimization system according to an embodiment of the present disclosure;

FIG. 2 is a schematic diagram illustrating the detailed structure of the assistant tool according to an embodiment of the present disclosure;

FIG. 3A is a schematic diagram illustrating a human-machine interface according to an embodiment of the present disclosure;

FIG. 3B to FIG. 3E are schematic diagrams illustrating the development mode of the assistant tool according to different embodiments of the present disclosure;

FIG. 3F is a schematic diagram illustrating the page of the assistant store according to an embodiment of the present disclosure;

FIG. 4A is a schematic diagram illustrating the use of the assistant tool according to an embodiment of the present disclosure;

FIG. 4B is a schematic diagram illustrating the use of the assistant tool according to another embodiment of the present disclosure; and

FIG. 5 is a flowchart illustrating the steps of a clinical workflow optimization method according to an embodiment of the present disclosure.

### DETAILED DESCRIPTION OF EMBODIMENT

Reference will now be made in detail to exemplary embodiments of the present disclosure, examples of which are illustrated in the accompanying drawings. Wherever possible, the same reference numerals are used in the drawings and description to refer to the same or like parts.

Throughout the specification and the appended claims, certain terms may be used to refer to specific components. Those skilled in the art will understand that electronic device manufacturers may refer to the same components by different names. The present disclosure does not intend to distinguish between components that have the same function but have different names. In the following description and claims, words such as “containing” and “comprising” are open-ended words, and should be interpreted as meaning “including but not limited to.”

The terms, such as “about”, “substantially”, or “approximately” are generally interpreted as within 10% of a given value or range, or as within 5%, 3%, 2%, 1% or 0.5% of a given value or range.

In the specification and claims, unless otherwise specified, ordinal numbers, such as “first” and “second”, used herein are intended to distinguish components rather than disclose explicitly or implicitly that names of the components bear the wording of the ordinal numbers. The ordinal numbers do not imply the order in which two components are in terms of space, time, or steps of a manufacturing method. Thus, what is referred to as a “first component” in the specification may be referred to as a “second component” in the claims.

In the present application, the terms “the given range is from the first numerical value to the second numerical value” and “the given range falls within the range from the first numerical value to the second numerical value” mean that the given range includes the first numerical value, the second numerical value, and other numerical values therebetween.

It is noted that the following are exemplary embodiments of the present application. However, the present disclosure is not limited thereto, while a feature of some embodiments can be applied to other embodiments through suitable modification, substitution, combination, or separation. In addition, the present disclosure can be combined with other known structures to form further embodiments.

Unless otherwise defined, all terms (including technical and scientific terms) used herein have the same meaning as commonly understood by those skilled in the art related to the present application. It can be understood that these terms, such as those defined in commonly used dictionaries, should be interpreted as having meaning consistent with the relevant technology and the background or context of the present disclosure, and should not be interpreted in an idealized or excessively formal way, unless there is a special definition in the embodiment of the present application.

In addition, the term “adjacent” in the specification and claims is used to describe mutual proximity and does not necessarily mean mutual contact.

In addition, descriptions such as “when” or “while” in the present application represent aspects such as “now, before, or after”, and are not limited to situations that occur at the same time. In the present application, similar descriptions such as “disposed on” refer to the corresponding positional relationship between the two components, and do not limit whether there is contact between the two components, unless otherwise specified. Furthermore, when the present disclosure provides multiple functions, if the word “or” is used between the functions, it means that the functions may exist independently. However, it does not exclude that multiple functions may exist simultaneously.

The various modules and units described herein may be implemented at least through hardware devices in combination with instructions, such as using a microprocessor to execute instructions stored in a non-transitory computer-readable medium in a computer program product to implement the functions of the various modules or units. The non-transitory computer-readable medium may be, for example, a hard drive, memory, a portable hard drive, a cloud server, or another hardware device with a data storage function, and is not limited thereto.

In the present disclosure, “communicative coupling” means that data may be transmitted between two devices using wired communication or wireless communication, and this means that there is a communication module between the two devices for realizing wired communication or wireless communication, while it is not limited thereto.

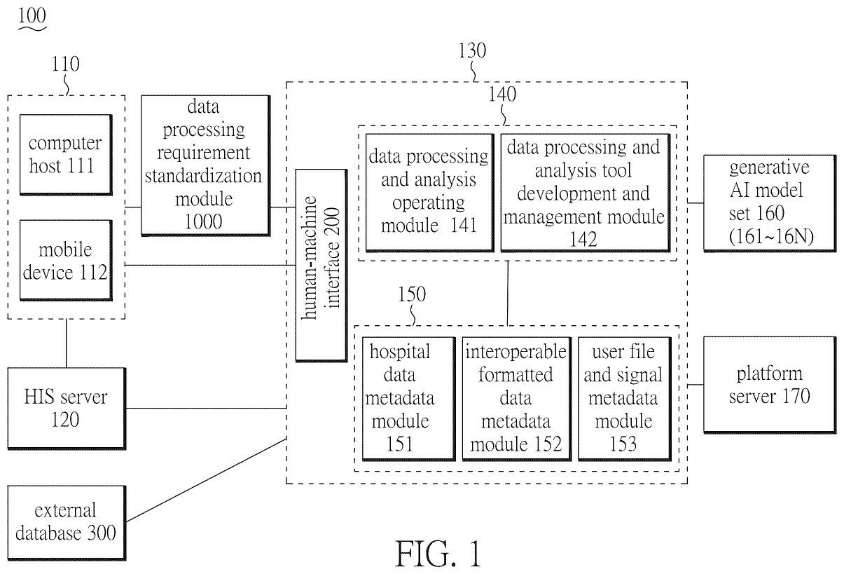

FIG. 1 is a system architecture diagram of a clinical workflow optimization system 100 according to an embodiment of the present disclosure. As shown in FIG. 1, the clinical workflow optimization system 100 may include a data processing and analysis platform 130 and a generative artificial intelligence (AI) model set 160. It may operate in conjunction with a user device 110, a hospital information system (HIS) server 120, and/or an external database 300. The data processing and analysis platform 130 may be communicatively connected with the user device 110, the hospital information system server 120, and the external database 300, and may provide one or more configured data processing requirement standardization modules (hereinafter referred to as assistant tool 1000) to the user device 110. For example, the data processing and analysis platform 130 may provide a human-machine interface 200 to display various functions of the platform and the assistant tool 1000, and the user may, for example, select the platform function or assistant tool 1000 to be used on the human-machine interface 200 through the user device 110. In one embodiment, the human-machine interface 200 may be presented to the user device 110 in the form of a web page or a software program. For example, the user may connect to the human-machine interface 200 in the form of a web page or may open the human-machine interface 200 in the form of a software program through the user device 110, while it is not limited thereto. The generative AI model set 160 may be electrically or communicatively connected to the data processing and analysis platform 130. The data processing and analysis platform 130 and the generative AI model set 160 may be installed on the same server or on different servers. The generative AI model set 160 may include at least one generative AI model 161˜16N, where N is a positive integer greater than or equal to 1. For ease of explanation, the following example assumes that the generative AI model set 160 includes a plurality of generative AI models 161˜16N. In one embodiment, at least a portion of the generative AI models 161˜16N are large language models, but it is not limited thereto.

In the present disclosure, the user device 110 may select one of the assistant tools 1000 to execute a task instruction (or known as a task target). The data processing and analysis platform 130 may interpret the content of the task instruction through one of the plurality of generative AI models 161˜16N (the content of the task instruction may be, for example, in natural language, but it is not limited thereto). After the content of the task instruction is interpreted, one or another of the generative AI models 161˜16N may execute the task instruction, wherein the data required to execute the task instruction may be recorded in the content of the task instruction, or may come from the user device 110 (for example, uploaded by the user device 110 to the data processing and analysis platform 130), or may come from the HIS server 120 (for example, the data processing and analysis platform 130 connects to the HIS server 120 to obtain the required data), or from the external database 300 (for example, the data processing and analysis platform 130 connects to a database of interoperable formatted data to obtain the required data), but it is not limited thereto. Upon completion, the data processing and analysis platform 130 may present the execution results of the task instruction to the user device 110, for example, through the human-machine interface 200 or the web page of the assistant tool 1000, but it is not limited thereto. The execution results of the task instruction may include, for example, information provision or message response, or other products that the AI models 161˜16N may provide, but are not limited thereto. In addition, in one embodiment, the assistant tool 1000 and the data processing and analysis platform 130 may also perform further processing on the execution results of the task instruction.

Regarding the user device 110, in one embodiment, the user device 110 may be, for example, a physical device used by a user to interact with the data processing and analysis platform 130. In one embodiment, the user device 110 may include a computer host 111 or a mobile device 112. The mobile device 112 may, for example, be any mobile device equipped with a microprocessor, such as a mobile phone, tablet computer, laptop computer, smart wearable device, or cloud device, but it is not limited thereto.

Regarding the HIS server 120, in one embodiment, the HIS server 120 may be, for example, a device within a medical institution (for example, a hospital) for storing patient data, clinical data, or medical knowledge-related data, but it is not limited thereto. Typically, a HIS system may be installed on the HIS server 120, and the HIS system may be used by personnel of the medical institution to access or manage various data stored on the HIS server 120. In one embodiment, each medical institution may have its own dedicated HIS system. That is, the HIS systems of different medical institutions may have varying standards for data format, storage methods, data management, data arrangement rules (such as, but not limited to, data arrangement rules), or data content arrangement methods. Therefore, under normal circumstances, data exchange between HIS systems of different hospitals is difficult. The data processing and analysis platform 130 of the present disclosure may be communicatively connected with multiple HIS servers 120 simultaneously. It may achieve data standardization or exchange between different HIS systems (to be described in the following paragraphs), which can solve the problems of the prior art.

Regarding the data processing and analysis platform 130, in one embodiment, the data processing and analysis platform 130 may be deemed as the center of the system, which is used to coordinate and integrate the operations of various related devices of the clinical workflow optimization system 100, but it is not limited thereto. In one embodiment, the data processing and analysis platform 130 may be set up on a platform server 170, wherein the various hardware devices in the platform server 170 (such as but not limited to microprocessors, controllers, hard drives, memories, communication modules, etc.) may be operated in conjunction with computer program products to realize the various functions of the data processing and analysis platform 130. In addition, in one embodiment, the generative AI model set 160 may be set on the platform server 170 of the data processing and analysis platform 130, but may also be set on other external servers.

In one embodiment, the data processing and analysis platform 130 may connect to the HIS server 120 or the external database 300 to obtain data or metadata from the HIS server 120 or the external database 300, or the user device 110 may upload data or metadata to the data processing and analysis platform 130. The storage device of the platform server 170 may be used to store or temporarily store such data or metadata. Here, “metadata” may refer to information such as the label or location of the data, rather than the actual content of the data, although it is not limited to this. In one embodiment, the types of data that the data processing and analysis platform 130 may obtain include hospital data, interoperable formatted data, and user-uploaded files. The hospital data may include clinical data, patient data, or medical documents from the medical system of a medical institution. The data format thereof may include, for example, data stored in a format corresponding to database format (data content or data format corresponds to database requirements), data in the form of documents (such as Word, PowerPoint, etc.), or data in the form of audio files, video files, or images, while it is not limited thereto. The interoperable formatted data may include, for example, clinical data, patient data, or medical document data that complies with interoperable formatted data specifications, such as, but not limited to, FHIR (Fast Healthcare Interoperability Resources) or LOINC (Logical Observation Identifiers Names and Codes). The user-uploaded files may include, for example, files uploaded by users via user device 110, such as, but not limited to, images, audio files, video files, or text files.

Furthermore, in one embodiment, the data processing and analysis platform 130 may include a user module 140 and a data source module 150.

The user module 140 may include a data processing and analysis operating module 141 and a data processing and analysis tool development and management module 142. The data source module 150 may include a hospital data metadata module 151, an interoperable formatted data metadata module 152, and a user file and signal metadata module 153, while it is not limited thereto.

The data processing and analysis operating module 141 may be used to control the various modules and generative artificial intelligence models 161˜16N in the data processing and analysis platform 130, or to execute specific algorithms for data processing or analysis. For example, the data processing and analysis operating module 141 may utilize the generative artificial intelligence models 161˜16N to convert data formats or standardize the arrangement of data content. Alternatively, the data processing and analysis operating module 141 itself may execute specific algorithms to convert data formats or standardize data content, while it is not limited thereto. Thus, the format or content of data from the HIS server 120 or the external database 300, or data uploaded by the user device 110, may be standardized through processing by the data processing and analysis operating module 141, or may be converted into standard-compliant interoperable formatted data. Therefore, through the data processing and analysis platform 130, data can be exchanged between HIS systems of different hospitals, thereby solving the problems of the prior art, while this is not limited thereto. Furthermore, the data processing and analysis tool development and management module 142 may be used to manage or develop the data processing and analysis platform 130. For example, platform developers may use the data processing and analysis tool development and management module 142 to execute various development tools for the data processing and analysis platform 130 and adjust various configuration parameters of the data processing and analysis platform 130, while it is not limited thereto. In one embodiment, various configuration parameters of the human-machine interface 200 may also be adjusted by the data processing and analysis tool development and management module 142, but it is not limited thereto.

The hospital data metadata module 151 may serve as a link between the data processing and analysis platform 130 and the HIS system of the HIS server 120, which is responsible for obtaining clinical data, patient data, or medical file data required by the data processing and analysis platform 130 from the HIS server 120 and may update the data on the HIS server 120, but it is not limited thereto. Alternatively, in one embodiment, the hospital data metadata module 151 may store metadata of the clinical data, patient data, or medical file data on the HIS server 120, but it is not limited thereto. The interoperable formatted data metadata module 152 may serve as a link between the data processing and analysis platform 130 and a server for storing or managing interoperable formatted data, which is responsible for obtaining interoperable formatted data from the interoperable formatted data server, or may obtain the specification information required to convert general data into interoperable formatted data, thereby facilitating data conversion by the data processing and analysis platform 130 or the generative AI models 161˜16N. Alternatively, the interoperable formatted data metadata module 152 may also store metadata for the interoperable formatted data, but this is not limited to this. The user file and signal metadata module 153 may be used to record metadata of data stored in a file format, such as the actual storage location of the file. Here, the file may include, but not be limited to, audio files, video files, or image files uploaded by the user through the user device 110.

Regarding the generative artificial intelligence model set 160, it may, for example, be a collection of a plurality of generative artificial intelligence models 161˜16N. In one embodiment, the plurality of generative artificial intelligence models 161˜16N may be, for example, large language models (LLMs) of different products or versions. Since the plurality of generative artificial intelligence models 161˜16N are large language models, the content of the task instructions issued by the user device 110 may be, for example, written in natural language, but it is not limited thereto. In one embodiment, various setting parameters of the generative artificial intelligence models 161˜16N may be adjusted by the data processing and analysis tool development and management module 142, but it is not limited thereto.

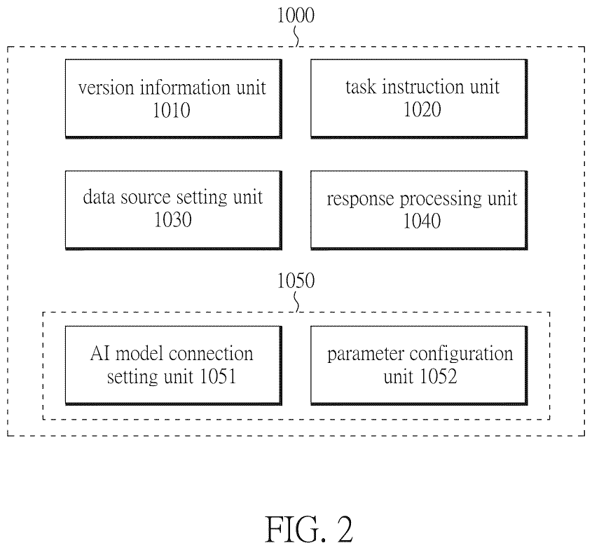

Regarding the assistant tool 1000, FIG. 2 is a schematic diagram illustrating the detailed structure of the assistant tool 1000 according to an embodiment of the present disclosure. Please refer to FIG. 1 for reference. As shown in FIG. 2, the assistant tool 1000 may include a version information unit 1010, a task instruction unit 1020, a data source setting unit 1030, a response processing unit 1040, an AI model connection setting unit 1051, and a parameter configuration unit 1052. The functions of these units may be implemented, for example, by a microprocessor executing instructions in a computer program product, while it is not limited thereto.

The assistant tool 1000 may be, for example, a tool program of the data processing and analysis platform 130. Each assistant tool 1000 may be connected to at least one AI model 160, and each assistant tool 1000 may have one or more preset task instructions. Therefore, each assistant tool 1000 may be considered an AI tool specifically for performing a specific task. In one embodiment, the assistant tool 1000 may correspond to a development mode and a usage mode. Basically, the development mode allows a user (for example, a developer) to create a new assistant tool 1000 or adjust the configuration of the assistant tool 1000, while the general usage mode allows the user to select the assistant tool 1000 to perform a task, which is not limited thereto.

In one embodiment, the version information unit 1010 of the assistant tool 1000 may be used to present version information of the assistant tool 1000, the data processing and analysis platform 130, and/or the generative artificial intelligence models 161˜16N, while it is not limited thereto.

In one embodiment, the task instruction unit 1020 of the assistant tool 1000 may be used to set the task instruction corresponding to the assistant tool 1000. For example, in development mode, the task instruction unit 1020 may display an input field on the human-machine interface 200. The developer may enter the content of a preset task instruction for the assistant tool 1000 in the input field, or the developer may use the editing function of the task instruction unit 1020 to adjust the content of the preset task instruction. In one embodiment, in the usage mode, the task instruction unit 1020 may also display an input field on the human-machine interface 200 for the user to enter an immediate task instruction or modify the content of the preset task instruction. Alternatively, the task instruction unit 1020 may also provide a data upload function on the human-machine interface 200, allowing the user to upload data, although this is not limited to this function. In one embodiment, the assistant tool 1000 may simultaneously have multiple preset task instructions, while it is not limited thereto.

In one embodiment, the data source setting unit 1030 of the assistant tool 1000 may be used to set the source of data related to the task instruction. For example, the developer may use the data source setting unit 1030 to set the source of data used by the assistant tool 1000 when executing the task instruction, such as uploaded by the user device 110, from the HIS server 120, or from the external database 300 (such as a server of interoperable formatted data or other database), etc., and may also be set not to provide data, while it is not limited thereto.

In one embodiment, the response processing unit 1040 of the assistant tool 1000 may be used to present the execution results of the task instruction. For example, the response processing unit 1040 may present the execution results of the AI models 161˜16N on the human-machine interface 200, while it is not limited thereto. In addition, the response processing unit 1040 may also allow the user to set subsequent processing plans for the execution results of the task instruction, such as outputting to a table, performing further analysis, providing feedback on the quality of the execution results, or writing the re-edited or formatted data back to the original database, etc., but it is not limited thereto.

In one embodiment, the AI model connection setting unit 1051 of the assistant tool 1000 is used to set one of the generative AI models 161˜16N used by the task instruction. For example, the developer may select the AI model 161˜16N used by the assistant tool 1000 when executing the task instruction through the AI model connection setting unit 1051. Different task instructions may set different AI models 161˜16N, while it is not limited thereto.

In one embodiment, the parameter configuration unit 1052 of the assistant tool 1000 may be used to set other parameters of the assistant tool 1000, such as the name, usage description, category, or set basic information such as the role played by the assistant tool 1000 (to improve the analysis efficiency of the AI model), while it is not limited thereto.

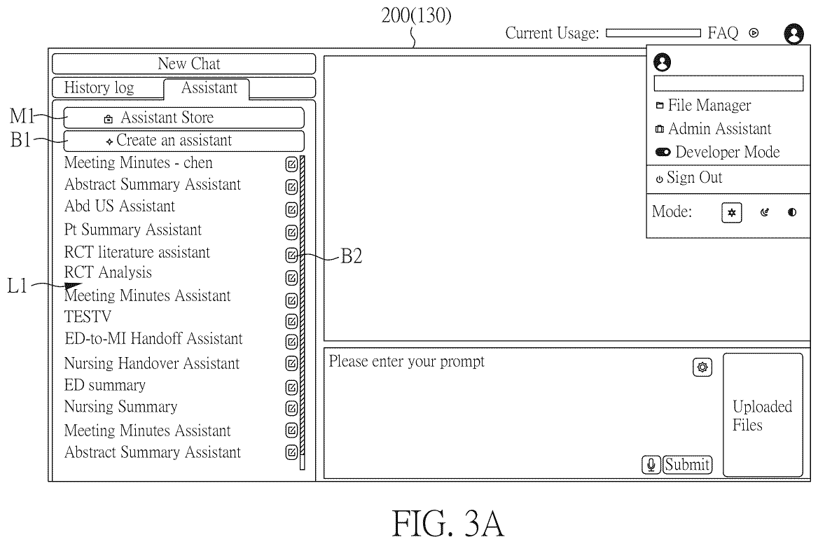

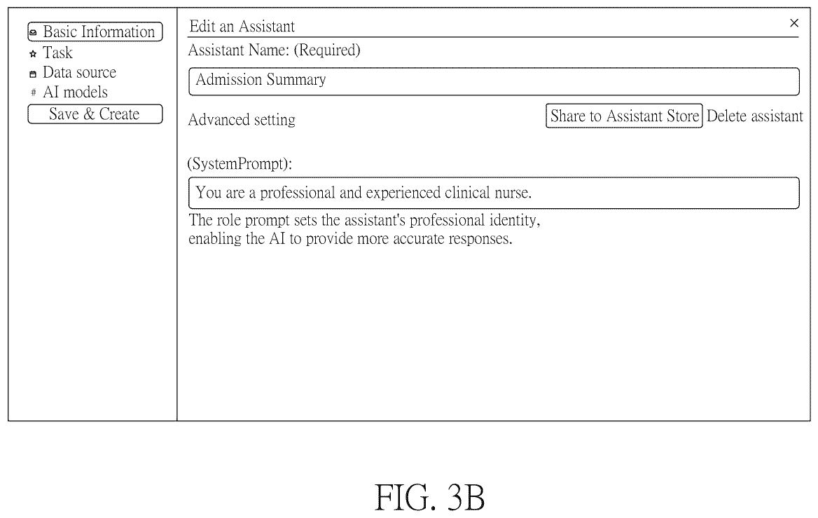

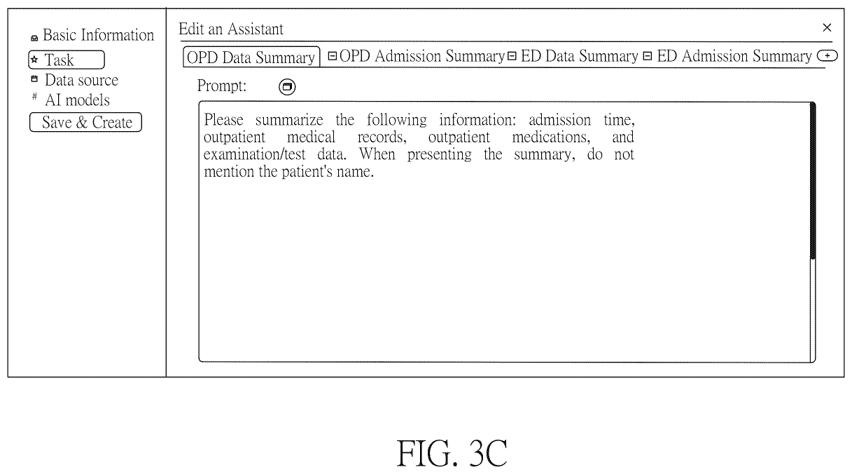

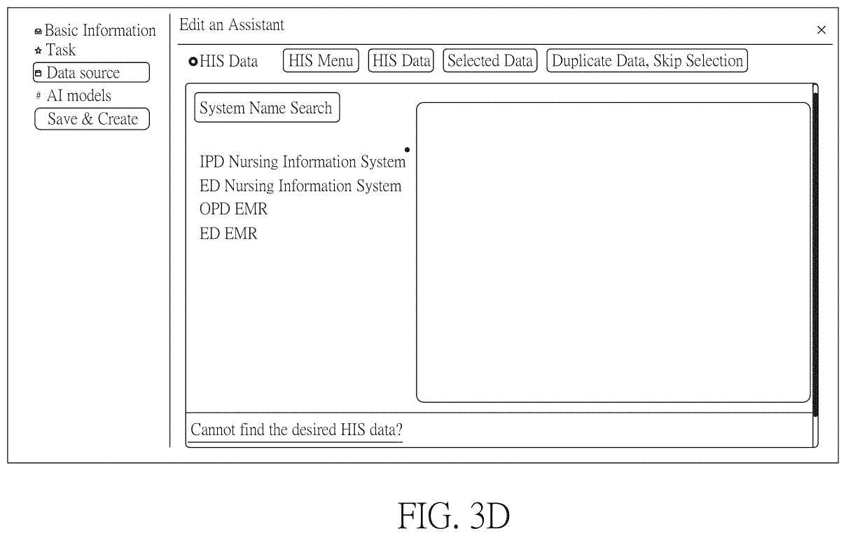

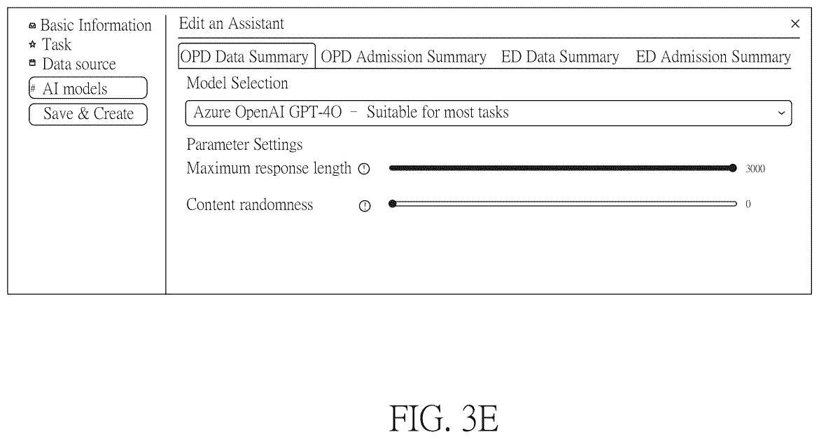

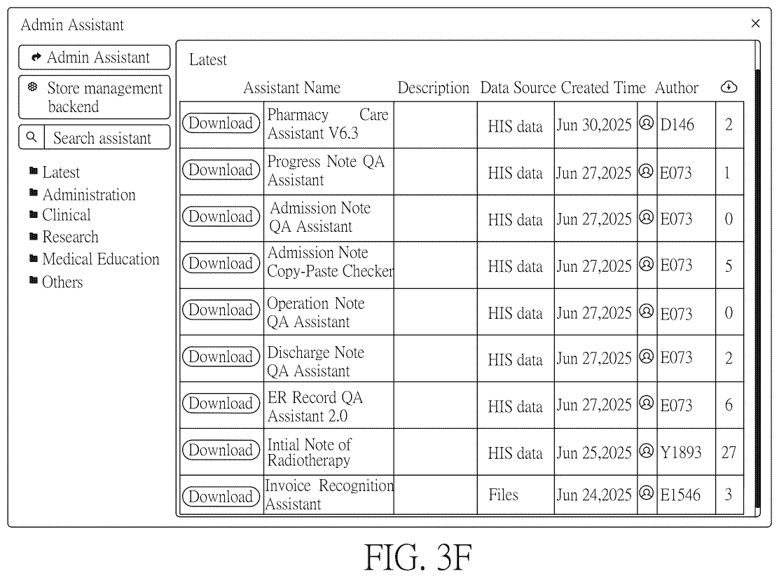

Next, the development mode of the assistant tool 1000 will be described using a practical example. FIG. 3A is a schematic diagram illustrating a human-machine interface 200 according to an embodiment of the present disclosure, FIG. 3B to FIG. 3E are schematic diagrams illustrating the development mode of the assistant tool 1000 according to different embodiments of the present disclosure, and please refer to FIG. 1 and FIG. 2 as a reference.

As shown in FIG. 3A, in development mode, when a developer connects to the data processing and analysis platform 130 via the user device 110, the human-machine interface 200 provided by the data processing and analysis platform 130 may display an interactive block B1 for creating an assistant tool 1000, or may display a list L1 of already created assistant tools 1000 and an interactive block B2 for editing already created assistant tools 1000. The developer may click on these interactive blocks (B1, B2) to create a new assistant tool 1000 or edit an already created assistant tool 1000. Furthermore, the human-machine interface 200 may also display an interactive block MI for an assistant store, which, when clicked, allows access to the page of the assistant store.

When entering the process of creating or editing the assistant tool 1000, a setting page for the assistant tool 1000 may be generated on the human-machine interface 200. The user may select basic settings, task target settings (i.e., task instruction settings), data source settings, or AI model settings, while it is not limited thereto. In other words, when entering the process of creating or editing the assistant tool 1000, the system 100 may be considered to provide a tool for creating the assistant tool 1000. The tool may provide a graphical or natural language interface for a user to set one or more elements independently, and the one or more elements include: data source and type, data screening conditions and pre-processing logic, the generative artificial intelligence model and execution parameters to be used, and the storage method of the processing results.

Regarding basic settings, as shown in FIG. 3B, on the basic settings page of the assistant tool 1000, the parameter configuration unit 1052 provides multiple fields for setting parameters of the assistant tool 1000, such as, but not limited to, setting the name, role prompt, or advanced settings of the assistant tool 1000. The above parameters are merely examples but not limitations, and may be increased or decreased as needed. Furthermore, on the basic settings page, the user may also choose whether to delete the assistant tool or share it with the assistant store, while this is not limited to these options.

Regarding task target settings, please refer to FIG. 3C. Each assistant tool 1000 may be provided with one or more task instructions (i.e., task targets)—for example, the assistant tool 1000 in FIG. 3C is provided with four task instructions: outpatient data collection, outpatient admission process, emergency data collection, and emergency admission process. However, more or fewer task instructions may be provided. These task instructions may correspond to different data sources (please refer to the subsequent paragraphs regarding FIG. 3D).

As shown in FIG. 3C, in the task target setting page, the task instruction unit 1020 may provide a field for the developer to write the task instruction (prompt) of the assistant tool 1000. Since the generative AI models 161˜16N may be large language models or include large language models, the task instruction written by the developer may be in natural language, while it is not limited thereto. In addition, in one embodiment, the execution mode of the task instruction may also be set. For example, it may be set to execute the task instructions set by the assistant tool 1000 in batch, and after the task instructions are completed, the respective results may be written back to the HIS system (for example, corresponding to three data fields), or it may be set to execute the task instructions set by the assistant tool 1000 simultaneously, merge the results of each task instruction, and then write them back to the HIS system (for example, corresponding to one data field), or it may first execute the task instructions set by the assistant tool 1000, then summarize all the results, and write the summary back to the HIS system (for example, corresponding to one data field), while it is not limited thereto.

The following are some examples of task instructions (i.e., task targets).

In one embodiment, the content of the task instruction may be, for example, “You are a natural language conversion system, and your purpose is to assist clinical nurses in converting oral content into daily ward rounds text records. Please follow the instructions below to complete the process and convert colloquial or specific pronunciation words into professional terms. Please convert specific pronunciations according to the following rules: for example, Nasal Canuula 3 L/min=Oxygen Nasal Cannula 3 L/min; Simple Mask=Simple Mask 6 L/min; and so on”. Then, when the task instruction is actually executed, at least the first one of the generative AI models 161˜16N may be used to interpret the content of the instruction, the second one of the generative AI models 161˜16N may be used to obtain relevant data from various data sources, and the third one of the generative AI models 161˜16N may be used actually to execute the content of the instruction, for example, it may be used in conjunction with voice recognition software or AI model to convert the user's voice into text, then convert specific words into professional terms, and generate a file of daily ward rounds text records, while it is not limited thereto. In addition, the first to third ones of the aforementioned generative AI models 161˜16N may be AI models with different capabilities, but may also be the same AI model, while it is not limited thereto.

In another embodiment, the content of the task instruction may be, for example, “You are an AI assistant specializing in assisting medical interpretation, and your task is to determine the degree of intestinal cleanliness of the patient before undergoing a colonoscopy. Please grade the provided toilet or bedpan photo as ‘excellent’, ‘acceptable’, or ‘poor’ based on the water quality and whether there is residual feces as the main judgment basis; and so on”. Then, when the task instruction is actually executed, at least the first of the generative AI models 161˜16N may be used to interpret the content of the instruction, and the second of the generative AI models 161˜16N may be used actually to execute the content of the instruction, for example, it will be used in conjunction with an image recognition AI model to analyze the bedpan or toilet image taken before the colonoscopy to assist in determining the patient's bowel cleansing preparation status.

In another embodiment, the content of the task instruction may be, for example, “You are a professional and experienced clinical nurse, and your task is to compile relevant clinical information of the patient before this hospitalization and write an admission history to describe the patient's reasons for admission, current condition, medications currently being taken, etc. First, please filter the patient's outpatient SOAP records before the “hospitalization time” and, based on the most recent SOAP that mentions the hospitalization plan, respectively extract the ‘Subjective’, ‘Objective’, and ‘Plan’, and attach a Chinese translation (in Traditional Chinese) below the original text. Second, according to this outpatient SOP, summarize the patient's reason for admission in Traditional Chinese, and so on” thereafter, when the task instruction is actually executed, at least a first one of generative AI models 161˜16N may be used to interpret the content of the instruction, a second one of generative AI models 161˜16N may be used to obtain relevant data from various data sources, and a third one of generative AI models 161˜16N may be used to actually execute the content of the instruction, such as extracting and compiling the clinical data mentioned in the instruction to generate a content description of the admission process.

In another embodiment, the content of the task instruction may be, for example, “You are a case quality review expert with emergency clinical experience. Please use your professional knowledge to analyze, score, and comment, item by item, on the emergency medical record content provided this time, based on the following evaluation criteria. The full score for each item is 5 points. Please give the following scores based on the completeness and compliance of the content: very consistent being 5 points, consistent being 4 points, acceptable being 3 points, non-compliant being 2 points, and very non-compliant being 0 points. If the review item does not apply to the medical record, please clearly mark “not applicable” and give a weighted score based on its impact on the overall medical record quality; and so on” Later, when the task instruction is actually executed, at least the first of generative AI models 161˜16N may be used to interpret the content of the instruction, while a second of generative AI models 161˜16N may be used to obtain relevant data from various data sources. A third of generative AI models 161˜16N may be used to actually execute the instruction, such as comparing original test results, examination reports, and other relevant clinical data, reviewing emergency medical records for omissions, generating emergency medical record review results, and providing a total score and improvement suggestions. The aforementioned task instruction contents are merely examples, but not limitations.

Regarding data source settings, please refer to FIG. 3D. On the data source settings page corresponding to each task instruction, a plurality of preliminary options are available for selection. These options include, but are not limited to, options for entering the HIS system menu, configuring HIS data, displaying selected HIS data, or notifying the system that the same data already exists and does not need to be selected again. Furthermore, on the data source settings page, keywords may be entered to search for HIS systems or HIS data that are desired as data sources for the task instructions, while it is not limited thereto.

Regarding AI model settings, each task instruction may also individually set the AI model to be used. Please refer to FIG. 3E. In the setting page of the AI model setting, the task instruction may be first selected, and then the AI model 161˜16N corresponding to the task instruction may be set, while it is not limited thereto. On the AI model setting page, the available AI models, ranging from 161 to 16N, may be presented in the form of a drop-down menu, but it is not limited thereto. In one embodiment, the setting page may also be used to set the parameters of the AI models 161˜16N, wherein the parameters may be, for example, but not limited to, the upper limit of the number of response words or the setting of content randomness, while it is not limited thereto.

In addition, when an assistant tool 1000 is created, the platform 130 may store the assistant tool 1000, and the developer may choose whether to make it public. For example, suppose it is chosen to be public. In the case, the platform 130 may display the assistant tool 1000 on the human-machine interface 200 for other users to select, while it is not limited thereto. In one embodiment, the clinical workflow optimization system 100 may provide an assistant store function, allowing the assistant tools 1000 that have been created and made public to be gathered and listed on the same page in a social sharing manner, thereby facilitating user selection. FIG. 3F is a schematic diagram illustrating the page of the assistant store according to an embodiment of the present disclosure. As shown in FIG. 3F, the page may list the names, function descriptions, data sources, creation time, authors, or download counts of all assistant tools 1000 in the assistant store, while it is not limited thereto, and users may select the assistant tool 1000 desired to be used. In addition, the assistant tools 1000 may also be classified according to categories, such as latest, system, administration, clinical, research, teaching, marketing, or other categories, while it is not limited thereto. It should be noted that the above are only examples and the content of the assistant store is not limited thereto.

Accordingly, the development model of the assistant tool 1000 can be understood.

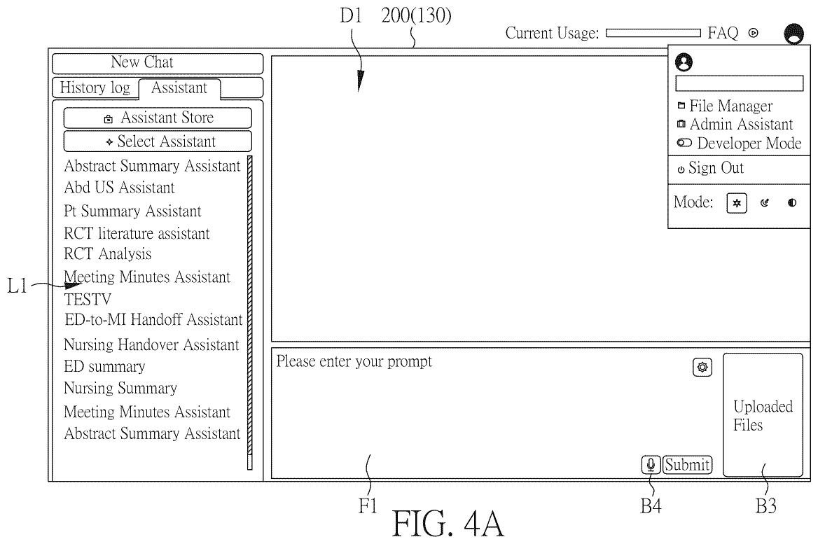

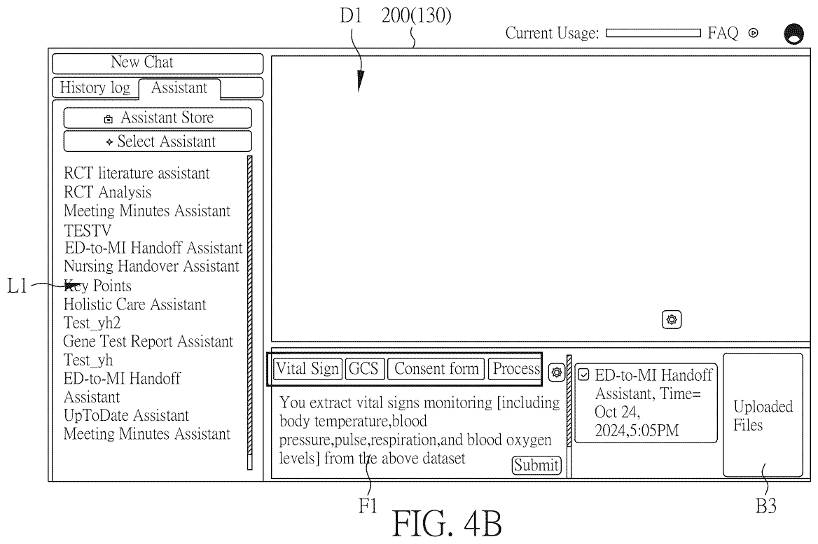

Next, the use of the assistant tool 1000 will be described using an actual example. FIG. 4A is a schematic diagram illustrating the use of the assistant tool 1000 according to an embodiment of the present disclosure, FIG. 4B is a schematic diagram illustrating the use of the assistant tool 1000 according to another embodiment of the present disclosure, and please refer to FIG. 1 to FIG. 3B for reference.

As shown in FIG. 4A, in the usage mode, when a user connects to the data processing and analysis platform 130 via the user device 110, the human-machine interface 200 provided by the data processing and analysis platform 130 may display a list L1 of created assistant tools 1000, allowing the user to select the assistant tool 1000 desired to be used. In one embodiment, the human-machine interface 200 may display a display area D1 (and an AI dialogue process) for displaying data such as the dialogue process between the user and the AI model, while it is not limited thereto. In other embodiments, the human-machine interface 200 may not display or have the display area D1. In one embodiment, the human-machine interface 200 may display an input field F1 and an interactive data upload block B3. The input field F1 allows the user to enter text, while the interactive data upload block B3 allows the user to upload data. Furthermore, in one embodiment, the human-machine interface 200 may also display an interactive block B4 for voice input and/or video input. When the user clicks on the interactive block B4, the voice input and/or video input function may be activated. Furthermore, when the user selects one of the assistant tools 1000 in the list L1, the input field F1 may also display the information (for example, task instructions) of the assistant tool 1000, while the present disclosure is not limited thereto.

As shown in FIG. 4B, when a user selects one of the assistant tools 1000 in the list L1, the content of the preset task instructions of the selected assistant tool 1000 may be displayed in the input field F1. In one embodiment, when the assistant tool 1000 has multiple preset task instructions, these preset task instructions may be displayed in a task selection field F2 for the user to select. Next, the user may click on the interactive block B3 to upload relevant data, and may click on the submit button to cause the assistant tool 1000 to begin executing the preset task instructions. In this case, the data processing and analysis platform 130 may use the generative AI models 161˜16N preset in the assistant tool 1000 to process the preset task instructions. In one embodiment, the user may also edit the content of the preset task instructions in the input field F1, or add additional immediate task instructions to the content of the preset task instructions, and then click on the confirmation button B5 to cause the assistant tool 1000 to begin executing the preset task instructions and the immediate task instructions. In addition, in one embodiment, the user may directly enter an immediate task instruction in the input field F1 without selecting any assistant tool 1000. In this case, the data processing and analysis platform 130 may assign one of the generative artificial intelligence models 161˜16N in a random or preset manner to process the user's immediate task instruction, while it is not limited thereto.

Accordingly, the usage mode of the assistant tool 1000 can be understood.

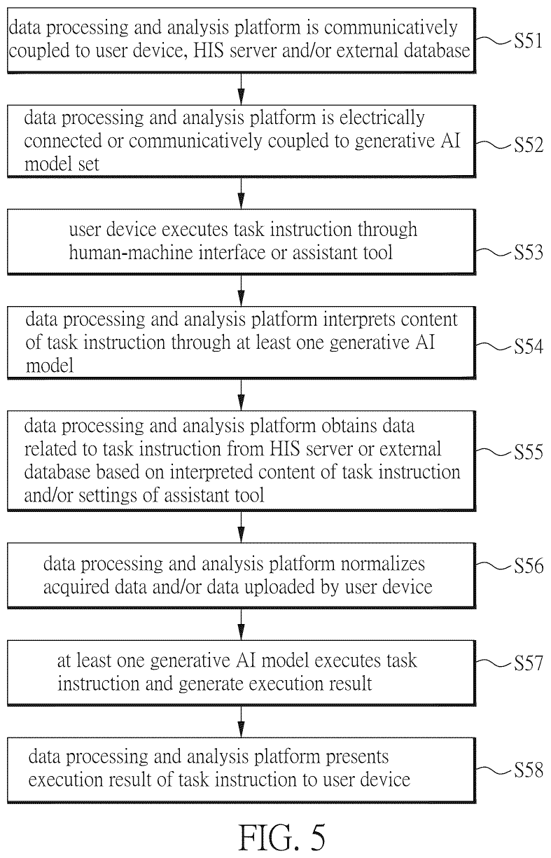

Next, the overall operation of the clinical workflow optimization system 100 will be described. FIG. 5 is a flowchart illustrating the steps of a clinical workflow optimization method according to an embodiment of the present disclosure, and please refer to FIG. 1 to FIG. 4B as reference. The clinical workflow optimization method is executed by the clinical workflow optimization system 100.

First, step S51 is executed, in which the data processing and analysis platform 130 is communicatively coupled to the user device 110, the HIS server 120, and/or the external database 300. Then, step S52 is executed, in which the data processing and analysis platform 130 is electrically connected or communicatively coupled to the generative AI model set 160. Then, step S53 is executed, in which the user device 110 executes a task instruction through the human-machine interface 200 or the assistant tool 1000. Then, step S54 is executed, in which the data processing and analysis platform 130 interprets the content of the task instruction through at least one of the generative AI models 161˜16N. Then, step S55 is executed, in which, based on the interpreted content of the task instruction and/or the settings of the assistant tool 1000, the data processing and analysis platform 130 may obtain data related to the task instruction from the HIS server 120 or the external database 300. Next, step S56 is executed, in which the data processing and analysis platform 130 normalizes the acquired data and/or the data uploaded by the user device 110. Next, step S57 is executed, in which at least one of the generative AI models 161˜16N executes the task instruction and generates an execution result. Next, step S58 is executed, in which the data processing and analysis platform 130 presents the execution result of the task instruction to the user device 110.

Regarding steps S51 and S52, the communicative coupling between the data processing and analysis platform 130 and the user device 110, the HIS server 120, the external database 300, or the generative AI set 160 may include a continuous connection, but may also include an idle or interrupted state where the connection may be restored at any time.

Regarding steps S53 and S54, the task instructions may include preset task instructions in the assistant tool 1000, real-time task instructions input by the user, or a combination thereof, while it is not limited thereto. In one embodiment, the generative AI models 161˜16N that interpret the task instructions may differ from the generative AI models 161˜16N that actually execute the task instructions, but they may also be the same.

Regarding step S55, in one embodiment, the data processing and analysis platform 130 may obtain metadata of clinical data related to the interpreted task instruction content through the hospital data metadata module 151, thereby obtaining the actual data from the HIS server 120. Alternatively, the data processing and analysis platform 130 may obtain metadata of interoperable formatted data related to the interpreted task instruction content through the interoperable formatted data metadata module 152, thereby obtaining the actual data from the external database 300. Alternatively, the data processing and analysis platform 130 may obtain the storage location of a user-uploaded file related to the task instruction content through the user file and signal metadata module 153, thereby obtaining the actual data. However, the present disclosure is not limited thereto.

Regarding step S56, in one embodiment, the data obtained by the data processing and analysis platform 130 from step S55 may be formatted or standardized using the generative AI models 161˜16N or algorithms inherent to the data processing and analysis platform 130, while it is not limited thereto. In one embodiment, when the data is audio, video or image, the data processing and analysis platform 130 may also convert the audio, video or image content into text data using an audio recognition, video recognition or image recognition algorithm, or may convert the audio, video or image content into text data using the generative AI models 161˜16N in conjunction with other AI models capable of recognizing audio, video, or image content, while it is not limited thereto.

Regarding step S57, in one embodiment, at least one of the generative AI models 161˜16N may actually execute the content of the task instructions, such as task instructions related to various medical system, including but not limited to data content organization, data content update, data content tabulation, data content analysis, data content integration, data content comparison, data content standardization, data content visualization, data content anomaly detection, data content multi-language processing, data content compliance checking, data content verification, data content export and report generation, etc. In one embodiment, the generative AI models 161˜16N used in each step may be the same or different.

Regarding step S58, in one embodiment, when the execution result of the task instruction is generated, the data processing and analysis platform 130 may notify the assistant tool 1000. The response processing unit 1040 of the assistant tool 1000 may display the execution result on the human-machine interface 200, or present the execution result on the page of the assistant tool 1000. In addition, in one embodiment, when subsequent processing is set, the response processing unit 1040 may also selectively match the generative AI model 161˜16N through the data processing and analysis platform 130 to perform subsequent processing operations and present the subsequent processing results to the user. In addition, in one embodiment, the processed data may also be written back to the data source. For example, the data processing and analysis platform 130 may update the metadata of the data or actually update the content of the data at the data source through the hospital data metadata module 151, the interoperable formatted data metadata module 152, or the user file and signal metadata module 153, while it is not limited thereto.

Accordingly, the method of optimizing clinical workflow can be understood.

The data processing and analysis platform 130 of the present disclosure may also have other functions or applications. In one embodiment, the data processing and analysis platform 130 may provide a generation quality assessment function, such as evaluating the execution results of a task instruction, wherein the generation quality assessment function may include at least one of four types: conventional execution generation quality response, questionnaire survey, natural language processing evaluation, and generative AI model evaluation. Regarding the “conventional execution generation quality response” type, after each task instruction execution result is presented, the generation quality assessment kit may automatically provide quality response options for the user to select, such as providing multiple evaluation levels for selection, while it is not limited thereto. Regarding the “questionnaire survey” type, the system developer may customize and create questionnaires and set users to receive the questionnaire survey. The data processing and analysis platform 130 may collect information from these questionnaires and output survey results. Then, the developer may perform statistical analysis, wherein the user can utilize the generative AI models 161˜16N to perform statistical analysis, while this is not limited to this approach. Regarding “natural language processing evaluation,” developers may use techniques such as BLEU (bilingual evaluation understudy), ROUGE (recall-oriented understudy for gisting evaluation), and/or METEOR (metric for evaluation of summaries) to assess generation quality. Regarding “generative AI model evaluation”, developers may create a dedicated assistant tool 1000 for evaluation to compare execution results with a golden dataset, while it is not limited thereto. The results of these evaluations may be fed back to the data processing and analysis platform 130 to improve the operations of the data processing and analysis platform 130 and the generative AI models 161˜16N.

In one embodiment, for the commonly used data of the hospital, the clinical workflow optimization system 100 may have a function similar to a toolbox, providing standardized data definitions and processing methods for all developers of the assistant tools 1000 in the hospital to use. For example, many developers need to organize patient medication records, so that the toolbox may provide preset task instructions on the organization method and output format of medication for developers to refer to and use. This may prevent developers from repeating the same work and may also standardize the format of medical records automatically completed by the system, thereby improving the quality of these records.

In one embodiment, to protect the developer's intellectual property rights, the clinical workflow optimization system 100 may have a function for specifying editing permissions for the assistant tool 1000. For example, when creating the assistant tool 1000, the developer may specify which users will have editing permissions for the assistant tool 1000. Authorized users may then edit and modify the assistant tool 1000. This allows the quality of the assistant tool 1000 to be optimized. For example, if a doctor, A, writes the first draft of the assistant tool 1000, then an authorized doctor, B, may supplement it based on their expertise. In addition, this function may also improve the application of the assistant tool 1000 among various units within the hospital. For example, units C and D both need to organize medical records. When unit C creates an assistant tool 1000, the authorized unit D may fine-tune the assistant tool 1000 according to needs to make the assistant tool 1000 more suitable for use in its own unit, thereby improving work efficiency and promoting collaboration and resource sharing among various units.

In one embodiment, if an assistant tool 1000 uses data in an interoperable data format, the assistant tool 1000 may add a call receiving and returning mechanism through encapsulation, for example, it may add an OAuth 2.0 authorization protocol, so that the assistant tool 1000 may provide services across medical institutions on the cloud, that is, the assistant tool 1000 may become software that may be used on the cloud, while it is not limited thereto.

In one embodiment, the clinical workflow optimization system 100 may avoid process debt in the HIS system. For example, the clinical workflow optimization system 100 and the HIS system are completely independent systems. The data extracted by the clinical workflow optimization system 100 from the HIS server 120 is processed on the data processing and analysis platform 130 without involving the operation of the HIS system. Therefore, the recurring interest of the process debt of the HIS system (such as continuously increasing maintenance costs, difficulty in integrating new technologies, etc.) may be avoided. In one embodiment, the clinical workflow optimization system 100 may handle the technical debt of the HIS system, that is, it may handle the different data formats and different specifications of the HIS system. For example, the clinical workflow optimization system 100 may convert data from different HIS systems into a standard format or into an interoperable data format. It may reorganize the data content from the user's perspective through the assistant tool 1000, thereby achieving the effect of cleaning and reconstructing the data of the HIS system, and thereby solving the technical debt problem of the HIS system. However, the present disclosure is not limited thereto.

From the above features, it can be seen that one of the advantages of the clinical workflow optimization system 100 of the present disclosure is to provide a simple and easy-to-use data processing and analysis platform 130. When medical personnel want to improve their workflow, they may easily create the assistant tool 1000 desired without the need to write any code. This can significantly improve the shortcomings of the existing mechanism, which only allows professional information personnel or manufacturers to develop single-function AI models.

Another advantage of the clinical workflow optimization system 100 of the present disclosure is that it may standardize clinical data processing, directly connect hospital databases and other data sources, and standardize data content through generative AI models 161˜16N. This can alleviate the problem of data being unable to communicate with each other across different hospital systems.

Another advantage of the clinical workflow optimization system 100 of the present disclosure is that the assistant tool 1000 may use clinical data in interoperable data formats such as FHIR and may be packaged into products such as SMART on FHIR, thereby creating a standalone product that may be used in the cloud. Therefore, the data processing and analysis platform 130 may be considered an incubator for products such as SMART on FHIR.

In addition, the clinical workflow optimization system 100 of the present disclosure has the following advantages: the system of the present disclosure adopts a human-machine collaborative architecture with human in the loop, and the user may modify the content of the system-generated response and send the modified response back to the data source, such as the HIS server 120. Alternatively, the user of the present disclosure not only creates an assistant tool 1000 that meets his/her own needs, but also shares the created assistant tool 1000 on the system to create a shared medical mutual aid community.

In summary, the effects of the present disclosure are summarized as follows: (1) the developer of the present disclosure may be a clinical business executive, and there is no need to create a dedicated AI tool in a programming language; (2) the data processing and analysis platform 130 may provide a generation quality assessment function to ensure the accuracy and consistency of the generated content of the AI model, which is suitable for clinical situations with high standards and high precision requirements; (3) it has the function of sharing the assistant tool 1000; (4) it adopts a human-machine collaboration architecture with human in the loop; (5) it may regularly evaluate the quality of the generated content of the AI model; (6) the assistant tool 1000 using clinical data in an interoperable data format such as FHIR may be packaged into an independent SMART on FHIR product; (7) the system has a standardized clinical data processing process, for example, it may first identify the content of the task instruction, then actually execute the task instruction through a specific AI model, and then return the execution result to the user; and (8) it may provide data flow that integrates various medical platforms, and may connect the medical platform with the generative AI model and multiple data sources to effectively execute the task instructions proposed by the user; (9) it may support diverse data sources; (10) users may provide data sources; (11) the AI model to be used is specified based on the task content; and (12) the data processing and analysis platform 130 may form an incubator for cross-medical institution products in the cloud.

Accordingly, the features and functions of the clinical workflow optimization system 100 of the present disclosure can be understood.

In one embodiment, the present disclosure may determine whether a product falls within the scope of protection of the present disclosure by at least determining the presence of a component in the product in context and/or the manner in which the component operates. If the content involves an algorithm, the algorithm of the product in the contest may also be used to determine whether the product falls within the scope of protection of the present disclosure, but it is not limited thereto. In one embodiment, the algorithm of the product in contest may be obtained, for example, through reverse engineering, but this is not limited to this method.

The details or features of the various embodiments of the present disclosure may be mixed and matched as needed, as long as they do not violate the spirit of the disclosure or conflict with each other.

Thus, the clinical workflow optimization system of the present disclosure may solve the problems of enormous pressure, low efficiency, or insufficient staff in the current nursing work environment.

The aforementioned specific embodiments should be construed as merely illustrative and not limiting the rest of the present disclosure in any way.

1. A clinical workflow optimization system, comprising:
a data processing and analysis platform communicatively coupled to a user device and a hospital information system server for providing one or more configured assistant tools; and
a generative artificial intelligence model set including at least one generative artificial intelligence model electrically connected or communicatively coupled to the data processing and analysis platform,
wherein, when the user device selects one of the assistant tools to execute a task instruction, the data processing and analysis platform interprets content of the task instruction through the at least one generative artificial intelligence model and executes the task instruction, and the data processing and analysis platform presents an execution result of the task instruction to the user device, wherein data required to execute the task instruction is recorded in the content of the task instruction, or comes from the hospital information system server, the user device or an external database.

2. The clinical workflow optimization system as claimed in claim 1, wherein the data processing and analysis platform includes a user module and a data source module, wherein the user module is provided to control the data source module and the generative artificial intelligence model, and wherein the data source module is provided to perform data transmission with the hospital information system server.

3. The clinical workflow optimization system as claimed in claim 2, wherein the data source module includes an interoperable formatted data metadata module, wherein the user module converts the data from the hospital information system server into a specific format through the interoperable formatted data metadata module, and wherein the data source module further includes a user file and signal metadata module for recording an actual location of the data stored in a file format.

4. The clinical workflow optimization system as claimed in claim 1, wherein the generative artificial intelligence model set includes a plurality of generative artificial intelligence models, the assistant tool includes a task instruction unit, a response processing unit, and an artificial intelligence model connection unit, the task instruction unit is provided to set one or more task instructions corresponding to the assistant tool, the response processing unit is provided to present execution results of the one or more task instructions or to set subsequent processing of the execution results, and the artificial intelligence model connection unit is provided to set one of the plurality of generative artificial intelligence models in the generative artificial intelligence model set used by the one or more task instructions.

5. The clinical workflow optimization system as claimed in claim 1, further comprising an assistant creation tool, which provides a graphical or natural language interface for a user to independently set one or more elements, wherein the one or more elements include: data source and type, data screening conditions and pre-processing logic, the generative artificial intelligence model and execution parameters to be used, and a storage method of the execution result.

6. A clinical workflow optimization method, executed by a clinical workflow optimization system including a data processing and analysis platform and a generative artificial intelligence model set provided with at least one generative artificial intelligence model, comprising the steps of:
communicatively coupling the data processing and analysis platform to a user device and a hospital information system server;
electrically connecting or communicatively coupling the data processing and analysis platform to the generative artificial intelligence model set;
enabling the data processing and analysis platform to provide one or more configured assistant tools;
when the user device selects one of the assistant tools to execute a task instruction, the data processing and analysis platform interpreting content of the task instruction through the at least one generative artificial intelligence model and executing the task instruction, wherein data required to execute the task instruction is recorded in the content of the task instruction, or comes from the hospital information system server, the user device or an external database; and
enabling the data processing and analysis platform to present an execution result of the task instruction to the user device.

7. The clinical workflow optimization method as claimed in claim 6, wherein the data processing and analysis platform includes a user module and a data source module, and wherein the user module is provided to control the data source module and the generative artificial intelligence model, and the data source module is provided to perform data transmission with the hospital information system server.

8. The clinical workflow optimization method as claimed in claim 7, wherein the data source module includes an interoperable formatted data metadata module, and the user module converts the data from the hospital information system server into a specific format through the interoperable formatted data metadata module, and wherein the data source module further includes a user file and signal metadata module for recording an actual location of the data stored in a file format.

9. The clinical workflow optimization method as claimed in claim 6, wherein the generative artificial intelligence model set includes a plurality of generative artificial intelligence models, the assistant tool includes a task instruction unit, a response processing unit and an artificial intelligence model connection unit, the task instruction unit is provided to set one or more task instructions corresponding to the assistant tool, the response processing unit is provided to present execution results of the one or more task instructions or to set subsequent processing of the execution results, and the artificial intelligence model connection unit is provided to set one of the plurality of generative artificial intelligence models in the generative artificial intelligence model set used by the one or more task instructions.

10. The clinical workflow optimization method as claimed in claim 6, wherein the clinical workflow optimization system further includes an assistant creation tool, which provides a graphical or natural language interface for a user to independently set one or more elements including: data source and type, data screening conditions and pre-processing logic, the generative artificial intelligence model and execution parameters to be used, and a storage method of the execution result.
